(19) United States
(12) Patent Application Publication
(10) Pub. No.: US 2026/0069733 A1
(43) Pub. Date: Mar. 12, 2026

(54) Instant-on Sanitizer with Ozone Generator

(71) Applicant: HEPCO HOLDINGS, LLC, St. Petersburg, FL (US)

(72) Inventor: Daniel J. Peterson, JR., Lakeland, FL (US)

(73) Assignee: HEPCO HOLDINGS, LLC, St. Petersburg, FL (US)

(21) Appl. No.: 18/828,708

(22) Filed: Sep. 9, 2024

(51) Int. Cl. A61L 2/26; A61L 2/08; A61L 2/10; A61L 2/20; A61L 2/24; A61L 101/02

(52) U.S. Cl. CPC A61L 2/26; A61L 2/084; A61L 2/10; A61L 2/202; A61L 2/24; A61L 2101/02; A61L 2202/11; A61L 2202/14; A61L 2202/15; A61L 2202/16

(57) ABSTRACT

A sanitization device for sanitizing a surface using ultraviolet light and ozone includes ultraviolet emitter(s) mounted to a board that is mounted to an enclosure such that each of the ultraviolet emitter(s) aims outwardly from the enclosure for radiating the surface. The board has at least one orifice for directing ozone onto the surface. There is an ozone generator for generating ozone from air and a fan that moves the ozone towards the board and through the orifice(s) to direct the ozone towards the surface. There is also a trigger switch electrically coupled to the ultraviolet emitter(s), to the ozone generator, and to the fan, responsive to an operation of the trigger switch, power is applied to the ultraviolet emitter(s), to the ozone generator, and to the fan thereby radiating the surface with the ultraviolet light and directing the ozone towards the surface.

### BACKGROUND OF THE INVENTION

It is known that the rising problem of antibiotic resistance has led to fears that medicine will return to the situation of a century ago when extensive wounds and surgery often led to death due to uncontrollable infection. These fears have in turn spurred a major research effort to find alternative antimicrobial approaches which, it is hypothesized, will kill resistant micro-organisms while being unlikely to cause additional resistance to develop. At the present time many international research efforts to discovery new antimicrobials are underway. Recently, the emphasis is on how to take precautions against creating, and if possible, eliminate multidrug resistance in concert with exploring new methods to kill pathogenic microorganisms. Karen et al. in “Tackling antibiotic resistance,” Bush K, Nat Rev Microbiol. 2011 Nov. 2; 9(12):894-6, pointed out that the investigation of novel non-antibiotic approaches, which can prevent and protect against infectious diseases should be encouraged, and should be looked upon as a high-priority for research and development projects.

One source of sterilization is UV-C radiation (wavelength: 200-280 nm). Among this wavelength range, the optimum range of 250-270 nm has the best potential ability to inactivate microorganisms because it this wavelength is absorbed by nucleic acids of microbial cells and, therefore is the most lethal range of wavelengths.

One way to reduce infection by resistant (or non-resistant) microorganisms is to neutralize as many of such organisms from the environment as possible. As some such organisms spread through the air, HEPA filters are often used to filter out such organisms before they infect humans, but many such organisms are transmitted on surfaces such as hands and shoes of people entering the area. To this, often before entering these areas, staff/patients must kill organisms from their hands using a germicidal compound or washing their hands. This helps keep microorganisms from spreading by way of hands, but what about the microorganisms that are on surfaces such as tables, clothing, instruments, and wounds?

Applying chemical products to such areas is partially effective. To kill or disable most pathogens, a very strong chemical is required. As the strength of the chemical increases, so does the risk of potential hazards to health and safety of both the people applying the chemical and to the users of the cleaned surfaces. This is not to mention issues related to allergies. Stronger chemicals tend to impact/discolor the surfaces on which they are applied, and many are not usable on a person. For example, bleach (chorine) is a known effective disinfectant, but bleach applied to clothing will result in decolorization and eventual decomposition. Furthermore, bleach (chlorine) does not kill many pathogens that have a protective shell (e.g., MRSA).

Beyond hospitals, many other areas are also prone to breed germs/microbes. For example, public showers in gyms, schools, etc.

UV radiation emitting devices (ultraviolet emitters or ultraviolet light bulbs) emit light with wavelengths of between, for example, 400-100 nm. Such ultraviolet light is known to kill at least a subset of known pathogens and, therefore, this light is suitable to reduce the number of pathogens on a surface.

Although ultraviolet light kills some pathogens and is suitable for that purpose, ultraviolet radiation alone is not effective in killing certain pathogens or classes of pathogens, especially pathogens that have protective envelopes or shells that protect the pathogens from the environment until the pathogens find their way into a suitable environment for growth, such as a wound. An example of such a pathogen is C-diff, which has a hard outer shell and is not significantly affected by UVC radiation. Bleach has been found effective in breaking this outer shell and killing C-diff, but bleach is impractical for use on many surfaces.

Ozone has been found to be effective in killing some pathogens that cannot be effectively killed with ultraviolet light alone. Ozone is a strong oxidizing agent that breaks through the encapsulation of some of the more difficult pathogens to kill such as C-diff. Ozone is effective in bacterial disinfection and the inactivation of many viruses.

Radiation emitting devices that do not have the surface treatment that filter out the 180 nm wavelengths are known and in use in other applications such as water sanitization, often known as germicidal lamps. These radiation emitting devices are usually mercury vapor tubes like typical fluorescent light bulbs but without any phosphor coating and without any material that impedes the passing of ultraviolet light, including ultraviolet light in the 253.7 wavelength range which is very good at destroying pathogens. These radiation emitting devices emit a broader range of ultraviolet that includes the 254 nm wavelength and also shorter wavelengths (e.g., less than 240 nm) that break the bond between dioxygen molecules (O₂+UV->2O), then the unstable oxygen atoms bond with another dioxygen molecule (O₂+O->O₃) forming ozone.

Unfortunately, these radiation emitting devices are costly, have limited bulb life, and do not immediately emit light once power is applied. There are now available light emitting diodes that emit ultraviolet light that have an expected life of at least four times that of mercury vapor tubes, but these ultraviolet light emitting diodes do not emit radiation at a wavelength that will produce significant ozone.

In handheld operation, the user typically aims the handheld device at an area that is to be sanitized (e.g., a table, clothing, instrument, wound) then initiates exposure to UV light by pressing a button/trigger. As many radiation emitting devices are mercury vapor gas discharge bulbs, there is often a considerable delay between energizing these radiation emitting devices and when sufficient UV radiation is emitted. For a handheld device, this delay is not desirable as the user (the person pressing the trigger) may not expose the area that is to be sanitized for sufficient time to achieve sufficient UV exposure for creating ozone and killing microbes.

Recently, LED emitters of ultraviolet light have become available. Such emitters have instant-on capabilities and emit light in the 200-280 nm range but are not capable of producing ozone.

What is needed is a handheld sanitization system that will provide fast-response UV light while emitting ozone for destruction of hard-to-kill microbes.

### SUMMARY OF THE INVENTION

Mechanisms are disclosed to emit ultraviolet light onto the target surface and generate ozone when a trigger is operated. The mechanisms include one or more ultraviolet emitting light emitting diodes that emit ultraviolet light and an ozone generator. In some embodiments, the ozone generator includes an electric arc and, in some such embodiments, a fan moves air that contains the ozone from the arc to the target surface.

In one embodiment, a sanitization device for sanitizing a surface using ultraviolet light and ozone is disclosed including at least one ultraviolet emitter mounted to a board. The board is mounted to an enclosure such that each of the ultraviolet emitter(s) aims outwardly from the enclosure for radiating the surface. The board has at least one orifice for directing ozone onto the surface. There is an ozone generator for generating ozone from air and a fan that moves the ozone towards the board and through the orifice(s) to direct the ozone towards the surface. There is also a trigger switch electrically coupled to the ultraviolet emitter(s), to the ozone generator, and to the fan, responsive to an operation of the trigger switch, power is applied to the ultraviolet emitter(s), to the ozone generator, and to the fan thereby radiating the surface with the ultraviolet light and directing the ozone towards the surface.

In another embodiment, a sanitization device for sanitizing a surface using ultraviolet light and ozone is disclosed including at least one ultraviolet emitter that is a light emitting diode. Rach of the ultraviolet emitter(s) is mounted to a board and the board is mounted to an enclosure such that each of the ultraviolet emitter(s) aims outwardly from the enclosure for radiating the surface. The board has at least one orifice for emitting ozone. An ozone generator is located within the enclosure for generating the ozone from air that enters through openings formed in the enclosure and a fan that is also mounted within the enclosure moves the ozone towards the board and through the at least one orifice to direct the ozone towards the surface. A trigger switch is electrically coupled to the ultraviolet emitter(s), to the ozone generator, and to the fan, such that, responsive to operation of the trigger switch, power is applied to the at least one ultraviolet emitter, to the ozone generator, and to the fan thereby radiating the surface with the ultraviolet light and directing ozone towards the surface.

In another embodiment, a sanitization device for sanitizing a surface using ultraviolet light and ozone is disclosed including at least one ultraviolet emitter that is a light emitting diode. Each ultraviolet emitter is mounted to a board and the board is mounted to an enclosure such that each ultraviolet emitter aims outwardly from the enclosure for radiating the surface. There is at least one orifice formed in the board. There is also at least one visible light emitter that is a visible light emitting diode. Each visible light emitter is mounted to the board such that each visible light emitter aims outwardly from the enclosure for radiating the surface. An ozone generator that produces an electric arc is located within the enclosure and generates ozone from air that enters through openings formed in the enclosure. A fan that is mounted within the enclosure moves air from the openings formed in the enclosure towards to the ozone generator and moves the ozone from the ozone generator through the at least one orifice in the board to direct the ozone towards the surface. There is a trigger switch that is electrically coupled to the ultraviolet emitter(s), to the visible light emitter(s), to the ozone generator, and to the fan, such that, responsive to operation of the trigger switch, power is applied to the ultraviolet emitter(s), to the visible light emitter(s), to the ozone generator, and to the fan thereby radiating the surface with the ultraviolet light and visible light and directing the ozone towards the surface.

### BRIEF DESCRIPTION OF DRAWINGS

The invention can be best understood by those having ordinary skill in the art by reference to the following detailed description when considered in conjunction with the accompanying drawings in which:

FIG. 1 illustrates a side-cutaway view of a sanitization device with an ozone generator.

FIG. 2 illustrates a bottom view of a sanitization device with an ozone generator.

FIG. 3 illustrates a front view of a sanitization device with an ozone generator.

FIG. 4 illustrates a schematic diagram of the handheld sanitization device with ozone generator.

FIG. 5 illustrates a schematic diagram of the handheld sanitization device.

FIG. 6 illustrates a flow diagram of the handheld sanitization device having the optional tilt sensor.

FIG. 7 illustrates a flow diagram of the handheld sanitization device without the optional tilt sensor.

### DETAILED DESCRIPTION OF THE INVENTION

Reference will now be made in detail to the presently preferred embodiments of the invention, examples of which are illustrated in the accompanying drawings. Throughout the following detailed description, the same reference numerals refer to the same elements in all figures.

Throughout the remainder of this description, the term “pathogen” will be used generically to denote any germ, virus, prion, fungus, spore, microbe, or other pathogen, capable or not capable of infecting a mammal such as a human.

Additionally, the described system is shown in detail for exposing a surface to ultraviolet light and ozone (O₃). There are many surfaces that are ideal for sanitizing using ultraviolet light and ozone including, but not limited to, tables, instruments, clothing, hands, wounds, floors, shoes, and tools. Note that there are known risks of exposing certain parts of a mammal's body to certain wavelengths of ultraviolet light, therefore, it is anticipated that proper precautions are taken to reduce exposure to such and, therefore, reduce such risks.

For brevity, various mechanical and electrical subcomponents such as supports, screws, wires, etc., are not described as such are well known in the art.

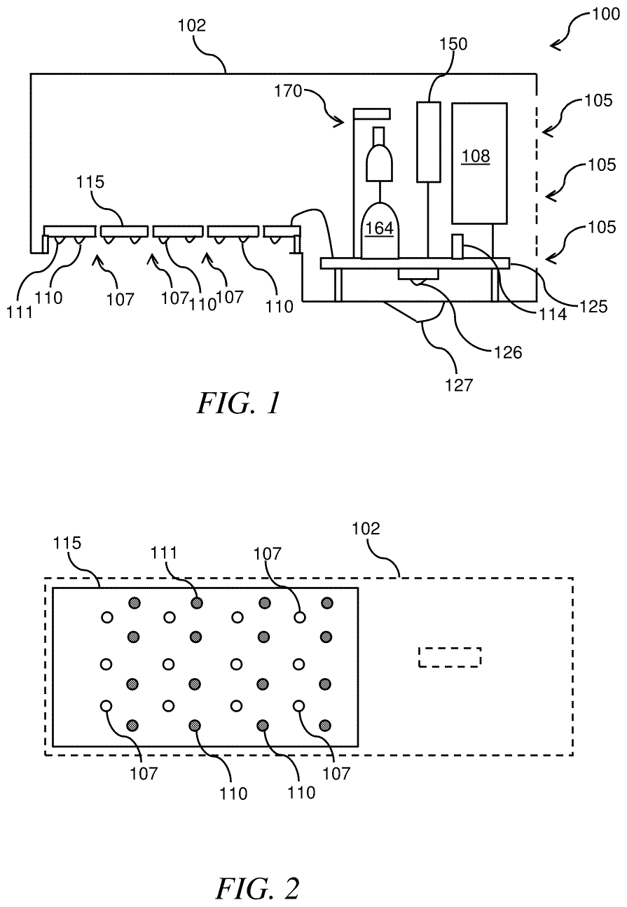

Referring to FIGS. 1, 2 and 3, views of a sanitization device 100 with an ozone generator 124 are shown. The sanitization device 100 is shown powered by a power source such as a battery 108, though any power source including AC line voltage is anticipated. Further, in some embodiments, the battery 108 is rechargeable and there is a connector (not shown for brevity and clarity reasons) on the enclosure 102 for connecting to a source of power for recharging, for example, a USB type connector.

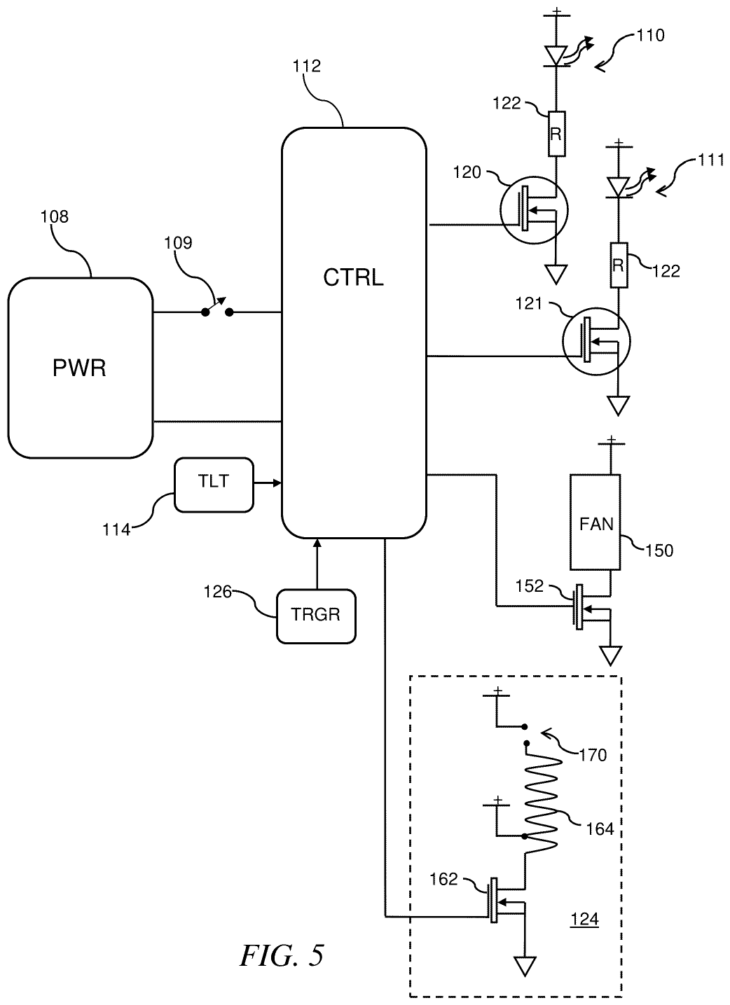

The sanitization device 100 has ultraviolet emitters 110 that emit ultraviolet light responsive to operation of the trigger switch 126 (see FIG. 5) that is mechanically interfaced to a trigger 127 (e.g., a button or other actuator that accepts mechanical force from a user). Note that, in some embodiments, there is a separate power switch 109 (see FIG. 5) that connects the power source (e.g., battery 108) to the circuitry that controls the ultraviolet emitters 110. In some embodiments, the trigger switch 126 connects the power source (e.g., battery 108) to the circuitry that controls the ultraviolet emitters 110 (e.g., light emitting diodes or LEDs) when it is activated. Note that in the latter, there is no need for a separate power switch 109, but nothing precludes having both the power switch 109 and the trigger switch 126. The ultraviolet emitters 110 are typically light emitting diodes that emit ultraviolet light which is known to destroy pathogens. The ultraviolet emitters 110 emit a range of ultraviolet that preferably includes the 254 nm wavelength (e.g., 253.7 nm). Since these ultraviolet emitters do not produce or emit ozone, a different source of ozone is needed. Ozone is generated by the ozone generator 124 that, in some embodiments includes an electric arc 170 that is powered by a high-voltage power conversion circuit. The electric arc 170 splits oxygen molecules (O₂) into single oxygen atoms (O). As the oxygen atoms are unstable, some oxygen atoms combine with oxygen molecules to form ozone atoms (O₃). The ozone is then moved (e.g., by a fan 15) to exit the enclosure 102 and onto the surface that is being disinfected to help destroying pathogens that are not easily destroyed solely with the ultraviolet light.

In some embodiments, the ultraviolet emitters 110 are mounted on a board 115 that has holes 107 through which the ozone escapes the enclosure 102. In some embodiments, vent holes 105 in the enclosure allow for air to enter the enclosure.

In the embodiment shown in FIG. 1, pressing the trigger 127 depresses the trigger switch 126 initiating operation of the ozone generator 124 that includes a flyback transformer 164 for generating sufficient voltage to create the electric arc 170.

In some embodiments, the circuit board 125 includes or is interfaced to a tilt sensing device 114. As ultraviolet light is often harmful to vision, the tilt sensor detects an orientation of the sanitization device 100 that might aim ultraviolet light towards the user, though in some embodiments, there is an override switch that will allow the sanitization device 100 to be used in an inverted configuration for sanitizing beneath objects such as tables, carts, and chairs.

In some embodiments there is one or more visible emitters 111. Although shown mounted to the board 115 on which the ultraviolet emitters 110 are mounted, there is no limitation as to the location of one or more visible emitters 111. In some such embodiments, the one or more visible emitters 111 are mounted on the board 115 to illuminate the surface that is to be disinfected. In some embodiments, the one or more visible emitters 111 are mounted on/through the enclosure 102 to provide a visible indication when the sanitization device 100 is operating.

In FIG. 2, the board 115 is shown with holes 107 for passing ozone out of the enclosure, several ultraviolet emitters 110 and one (optional) visible light emitter 111.

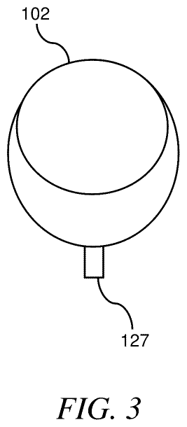

In FIG. 3, a front view of one physical embodiment of the enclosure is shown, though any physical enclosure is anticipated.

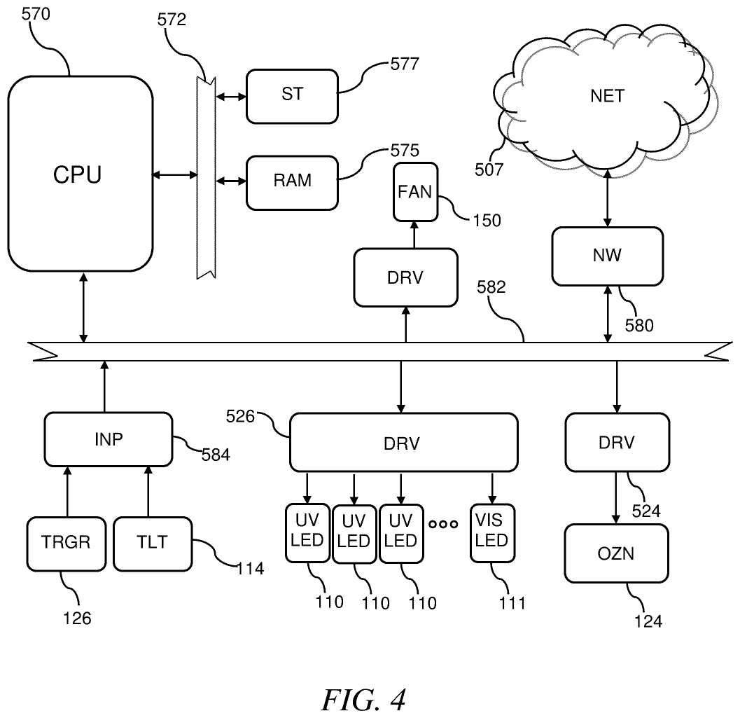

Referring to FIG. 4, a schematic diagram of the sanitization device 100 is shown. In this embodiment, the circuit board 125 includes a processor or microcontroller, referred to as a CPU 570. Interfaced to the CPU 570 is data storage 577 for storing programs, etc., and random-access memory 575. In some embodiments, the data storage and random-access memory 575 are interfaced to the CPU by a memory bus 572 or any way known in the industry (e.g., internal storage). In some embodiments, there is a system bus 582 for connecting to input/output subsystems, though it is fully anticipated that the CPU 570 is a microcontroller having internal input/output circuitry. In such embodiments, the trigger switch 126 and optional tilt sensor 114 are interfaced to an input port 584 that is either internal to the CPU 570 or interfaced by way of an input port 584 interfaced to the system bus 582. The ultraviolet emitters 110 and optional visible light emitters 111 are controlled by a driver 526 that is either internal to the CPU 570 or interfaced to the system bus 582. The ozone generator 124 is interfaced to a second driver 524 that is either internal to the CPU 570 or interfaced to the system bus 582.

In some embodiments, a wireless interface 580 is provided for communication wirelessly through a network 507 to report on activity and to signal any failures, such as failure of one of the ultraviolet emitters 110. When the wireless interface 580 is provided, it is also anticipated that, in some embodiments, the sanitization device 100 is enabled/disabled through the network 507.

Referring to FIG. 5, a schematic diagram of one embodiment of the sanitization device 100 is shown. Central to this embodiment is a control circuit 112 that receives power from the power supply (e.g., battery 108). In this embodiment, there is a power on/off switch 109 that turns off power to the sanitization device 100, for example, disabling the sanitization device 100 when not used for extended periods of time. In this example, the ultraviolet emitters 110 are controlled by a switching transistor 120 and are provided with the requisite power through a current limiting resistor 122. Likewise, the visible emitters 111, when present, are controlled by a switching transistor 121 and are provided with the requisite power through a current limiting resistor 122.

The fan 150 is controlled by another switching transistor 152 (on/off), though other arrangements are fully anticipated to control the fan 150 at variable speeds.

The ozone generator 124 is controlled by another switching transistor 162. In this example, a pulse waveform is applied to the base of the switching transistor 162. During the peak of the pulse, the smaller number of turns of the winding of the flyback transformer 164 saturate a core of the flyback transformer 164. When the pulse abates, the magnetic field collapses and a high-voltage pulse is emitted from the larger number of turns of the flyback transformer 164. This high-voltage pulse causes an electric arc 170 between two electrodes for creation of ozone gas.

In embodiments having a separate trigger switch 126, that trigger switch 126 is interfaced to the control circuit 112.

In embodiments having a tilt sensor 114, the tilt sensor 114 is also interfaced to the control circuit 112.

It is anticipated that the control circuit includes logic and/or a processor to receive inputs from the trigger switch 126 and tilt sensor 114 (when present) and to properly control the switching transistors 120/121/152/162.

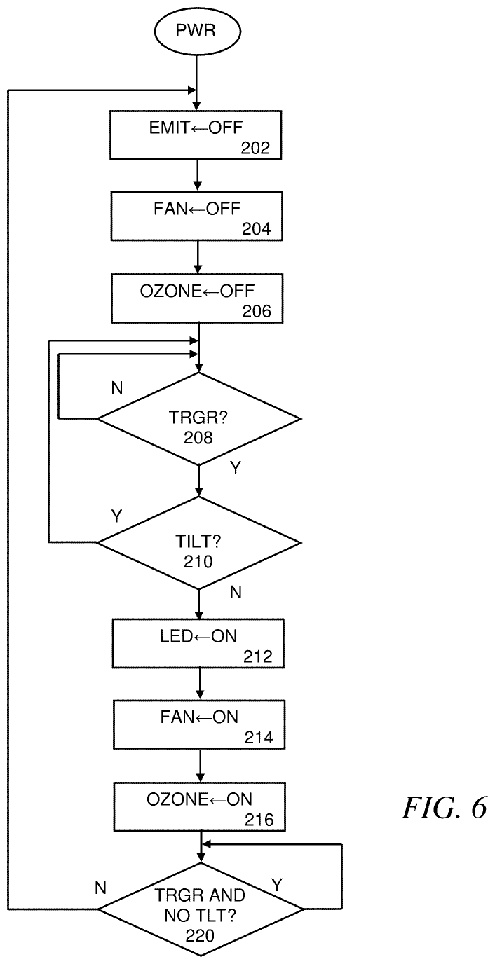

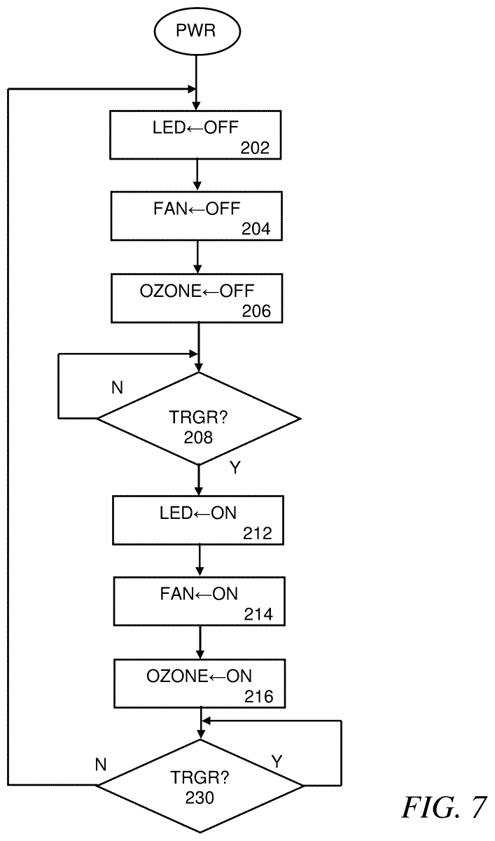

Referring to FIGS. 6 and 7, flow diagrams of the sanitization device 100 are shown. FIG. 6 shows a typical program flow of the sanitization device 100 with the optional tilt sensor. After power is applied to the system, power is disconnected 202 to all emitters, e.g., unbiasing the switching transistors 120/121. Likewise, power is disconnected 204 from the fan, e.g., unbiasing the switching transistor 152 and power is disconnected 206 from the ozone generator 124, e.g., unbiasing the switching transistor 162.

Now, a loop continues until it is detected 208, that the trigger switch 126 is activated. After it is detected that the trigger switch 126 is activated, the tilt sensor 114 is checked and if the tilt sensor 114 indicates improper orientation 210 of the sanitization device 100, the loop continues. If the tilt sensor 114 indicates proper orientation 210 of the sanitization device 100, power is connected 212 to all emitters, e.g., biasing the switching transistors 120/121.

Likewise, power is connected 214 to the fan 150, e.g., biasing the switching transistor 152 and power is connected 216 to the ozone generator 124, e.g., biasing the switching transistor 162. Power continues to the emitters 110/111, the fan 150, and the ozone generator 124 until either the tilt sensor 114 indicates improper orientation or until the trigger switch 126 is deactivated 220, at which time the above is repeated.

FIG. 7 shows a typical program flow of the sanitization device 100 without the optional tilt sensor. After power is applied to the system, power is disconnected 202 to all emitters, e.g., unbiasing the switching transistors 120/121. Likewise, power is disconnected 204 from the fan, e.g., unbiasing the switching transistor 152 and power is disconnected 206 from the ozone generator 124, e.g., unbiasing the switching transistor 162.

Now, a loop continues until it is detected 208, that the trigger switch 126 is activated. After it is detected that the trigger switch 126 is activated, power is connected 212 to all emitters, e.g., biasing the switching transistors 120/121. Likewise, power is connected 214 to the fan 150, e.g., biasing the switching transistor 152 and power is connected 216 to the ozone generator 124, e.g., biasing the switching transistor 162. Power continues to the emitters 110/111, the fan 150, and the ozone generator 124 until the trigger switch 126 is deactivated 230, at which time the above is repeated.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The form herein before described being merely exemplary and explanatory embodiment thereof. It is the intention of the following claims to encompass and include such changes.

1. A sanitization device for sanitizing a surface using ultraviolet light and ozone, the sanitization device comprising:
at least one ultraviolet emitter mounted to a board, the board mounted to an enclosure such that each of the at least one ultraviolet emitter aims outwardly from the enclosure for radiating the surface, the board having at least one orifice;
an ozone generator for generating the ozone from air;
a fan for moving the ozone towards the board and through the at least one orifice to direct the ozone towards the surface; and
a trigger switch electrically coupled to the at least one ultraviolet emitter, to the ozone generator, and to the fan, responsive to an operation of the trigger switch, power is applied to the at least one ultraviolet emitter, to the ozone generator, and to the fan thereby radiating the surface with the ultraviolet light and directing the ozone towards the surface.

2. The sanitization device of claim 1, wherein the ozone generator produces the ozone from oxygen in the air by way of an electric arc.

3. The sanitization device of claim 1, wherein the ozone generator comprises a flyback transformer, upon electrical pulses provided to an input winding of the flyback transformer, an output winding of the flyback transformer, being connected to a contact, produces high voltage pulses that create an arc between the contact and a second contact that is connected to a low-voltage potential or a round potential.

4. The sanitization device of claim 1, wherein the at least one ultraviolet emitter is a light emitting diode that emits light of a wavelength between 200 nm and 280 nm.

5. The sanitization device of claim 1, wherein the at least one ultraviolet emitter is a light emitting diode that emits light in a 254 nm wavelength.

6. The sanitization device of claim 1, further comprising a tilt sensor, the tilt sensor configured to inhibit the operation of the at least one ultraviolet emitter when the sanitization device is oriented upward, thereby, preventing emission of the ultraviolet light when the sanitization device is oriented upward.

7. The sanitization device of claim 1, wherein the trigger switch signals an electronic circuit within the enclosure to provide the power to the at least one ultraviolet emitter, the ozone generator, and the fan.

8. A sanitization device for sanitizing a surface using ultraviolet light and ozone, the sanitization device comprising:
at least one ultraviolet emitter that is a light emitting diode, each of the at least one ultraviolet emitter is mounted to a board and the board is mounted to an enclosure such that each of the at least one ultraviolet emitter aims outwardly from the enclosure for radiating the surface, the board having at least one orifice;
an ozone generator located within the enclosure for generating the ozone from air that enters through openings formed in the enclosure;
a fan mounted within the enclosure for moving the ozone towards the board and through the at least one orifice to direct the ozone towards the surface; and
a trigger switch electrically coupled to the at least one ultraviolet emitter, to the ozone generator, and to the fan; responsive to operation of the trigger switch, power is applied to the at least one ultraviolet emitter, to the ozone generator, and to the fan thereby radiating the surface with the ultraviolet light and directing ozone towards the surface.

9. The sanitization device of claim 8, wherein the ozone generator produces the ozone from oxygen in the air by way of an electric arc.

10. The sanitization device of claim 8, wherein the ozone generator comprises a flyback transformer, upon electrical pulses provided to an input winding of the flyback transformer, an output winding of the flyback transformer, being connected to a contact, produces high voltage pulses that create an arc between the contact and a second contact that is connected to a low-voltage potential or a round potential.

11. The sanitization device of claim 8, wherein the at least one ultraviolet emitter emits light of a wavelength between 200 nm and 280 nm.

12. The sanitization device of claim 8, wherein the at least one ultraviolet emitter emits light in a 254 nm wavelength.

13. The sanitization device of claim 8, further comprising a tilt sensor, the tilt sensor configured to inhibit the operation of the at least one ultraviolet emitter when the sanitization device is oriented upward, thereby, preventing emission of the ultraviolet light when the sanitization device is oriented upward.

14. The sanitization device of claim 8, wherein the trigger switch signals an electronic circuit within the enclosure to provide the power to the at least one ultraviolet emitter, the ozone generator, and the fan.

15. The sanitization device of claim 8, further comprising at least one visible light emitter that is a visible light emitting diode, each of the at least one visible light emitter is mounted to the board or to the enclosure.

16. The sanitization device of claim 15, wherein the at least one visible light emitter is mounted to the board such that visible light is directed toward the surface during the operation of the trigger switch.

17. A sanitization device for sanitizing a surface using ultraviolet light and ozone, the sanitization device comprising:
at least one ultraviolet emitter that is a light emitting diode, each of the at least one ultraviolet emitter is mounted to a board and the board is mounted to an enclosure such that each of the at least one ultraviolet emitter aims outwardly from the enclosure for radiating the surface, the board having at least one orifice;
at least one visible light emitter that is a visible light emitting diode, each of the at least one visible light emitter is mounted to the board such that each of the at least one visible light emitter aims outwardly from the enclosure for radiating the surface;
an ozone generator comprising an electric arc, the ozone generator located within the enclosure for generating the ozone from air that enters through openings formed in the enclosure;
a fan mounted within the enclosure for moving the air from the openings formed in the enclosure towards to the ozone generator and moving the ozone from the ozone generator through the at least one orifice in the board to direct the ozone towards the surface; and
a trigger switch electrically coupled to the at least one ultraviolet emitter, coupled to the at least one visible light emitter, coupled to the ozone generator, and coupled to the fan; responsive to operation of the trigger switch, power is applied to the at least one ultraviolet emitter, to the at least one visible light emitter, to the ozone generator, and to the fan thereby radiating the surface with the ultraviolet light and visible light and directing the ozone towards the surface.

18. The sanitization device of claim 17, wherein the ozone generator comprises a flyback transformer, upon electrical pulses provided to an input winding of the flyback transformer, an output winding of the flyback transformer, being connected to a contact, produces high voltage pulses that create an arc between the contact and a second contact that is connected to a low-voltage potential or a round potential.

19. The sanitization device of claim 17, wherein the at least one ultraviolet emitter emits light of a wavelength between 200 nm and 280 nm.

20. The sanitization device of claim 17, wherein the at least one ultraviolet emitter emits light in a 254 nm wavelength.
